(19) United States
(12) Patent Application Publication
(10) Pub. No.: US 2020/0262727 A1
(43) Pub. Date: Aug. 20, 2020

(54) Method for determining optimal preservation temperature of aerobic denitrifiers in wastewater treatment for total nitrogen removal

(71) Applicant: Jiangnan University, Wuxi (CN)

(72) Inventors: Shuo WANG, Wuxi (CN); Yan SHI, Wuxi (CN); Hui LV, Wuxi (CN); Ji LI, Wuxi (CN); Yan WANG, Wuxi (CN)

(21) Appl. No.: 16/869,670

(22) Filed: May 8, 2020

Related U.S. Application Data
(63) Continuation of application No. PCT/CN2019/102713, filed on Aug. 27, 2019.

(30) Foreign Application Priority Data
Oct. 30, 2018 (CN) 2018112772330

(51) Int. Cl. C02F 3/34; C02F 3/12; C12Q 1/04; G01N 15/14

(52) U.S. Cl. CPC C02F 3/348; C02F 3/1205; C02F 2101/16; G01N 15/1404; C12Q 1/04

(57) ABSTRACT

The present disclosure relates to a method for determining optimal preservation temperature of aerobic denitrifiers in wastewater treatment for total nitrogen removal, and belongs to the technical field of environmental engineering. The method of the present disclosure comprises measuring the cell activity state of the aerobic denitrifier stored at different temperatures based on flow cytometry, and taking the preservation temperature closest to the cell activity state of the aerobic denitrifier during the pilot operation as the optimum preservation temperature, and the data obtained from the test uses the cell activity state and performance effects after activity recovery to verify reliability. By using the method of the present disclosure, the step of recovering the aerobic denitrifier activity can be omitted, and the wastewater treatment plant, which intends to adopt the aerobic denitrifier process technology to achieve efficient removal of nitrate and total nitrogen, is effectively helped to realize the energy saving, consumption reducing operation, and the removal rate of nitrate and total nitrogen can reach 90% and 88% respectively. At the same time, the starting time of engineering application of the aerobic denitrifier process can be effectively shortened, the long-term stable operation of the aerobic denitrifier process is maintained, and the method has high industrial feasibility.

### TECHNICAL FIELD

The present disclosure relates to a method for determining optimal preservation temperature of aerobic denitrifiers in wastewater treatment for total nitrogen removal, and belongs to the technical field of environmental engineering.

### BACKGROUND

The stable and efficient removal of total nitrogen has always been the focus and difficulty of the operation of sewage treatment plants. With the increasing discharge standards of sewage treatment, it is particularly important to develop and promote the application of suitable nitrogen removal technologies. Due to the limitation of land for sewage treatment plants, adding carbon sources substances to anoxic tanks has become a method for most sewage treatment plants to improve the total nitrogen removal efficiency, but this method will not only significantly increase the cost of sewage treatment, but also cause sewage slowly degrade organic matter and waste the carbon source in the sludge. Therefore, wastewater denitrification technology based on land saving goals and zero external carbon source has been paid more and more attention. Aerobic denitrification refers to the process in which aerobic denitrifiers convert higher concentrations of nitrate into nitrogen gas in aerobic tanks through perplasmic nitrate reductases. This process does not require an additional carbon source substances, only the polyurethane filler enriched with aerobic denitrifiers needs to be added to the aerobic tank. Through the catalysis of periplasmic nitrate reductase, the total nitrogen in the sewage treatment plant can reach a stable standard emissions and energy saving operation.

Aerobic denitrifiers have good denitrification ability, and are generally attached to polyurethane suspension filler for growth to prevent it from being discharged with the aerobic tank effluent. If the polyurethane filler enriched with the cultured mature aerobic denitrifier should be used to overcome the difficulty of achieving total nitrogen standards. Sewage treatment plants with limited land can not only effectively shorten the time for the total nitrogen discharge of the sewage treatment plants, but also effectively save carbon source materials.

### SUMMARY

In order to simplify the activity recovery process of the aerobic denitrifier, allow the pollutant indexes of the wastewater treatment plant to reach the standard for discharge in a short time and achieve the land saving, energy saving and consumption reducing effects at the same time, the present disclosure characterizes cell activity states in the aerobic denitrifier stored under different temperature conditions based on the flow cytometry, verifies the characterization result of the flow cytometry according to aerobic denitrifiers activity recovery effect and cell activity status after microbial activity recovery, and finally establishes a method for determining the optimum preservation temperature of aerobic denitrifiers based on the flow cytometry, and provides technical support for high-standard pollutant discharge and energy saving and consumption reducing operation of the wastewater treatment plant.

A first object of the present disclosure is to provide a method for determining an optimum preservation temperature of aerobic denitrifiers, which comprises measuring the cell activity state of the aerobic denitrifier stored at different temperatures based on flow cytometry, and taking the preservation temperature closest to the cell activity state of the aerobic denitrifier during the pilot operation as the optimum preservation temperature. The measurement of the cell activity state of the aerobic denitrifier comprises the measurement of the content of living cells, early apoptotic cells, late apoptotic cells and dead cells.

In one embodiment of the present disclosure, the step of determining the optimum temperature in the flow cytometry comprises:

(1) Preparing an aerobic denitrifier test sample solution: diluting an aerobic denitrifier sample with a buffer, mixing evenly, filtering for precipitation, centrifuging, leaving the supernatant, purging the cells with a pre-cooled phosphate buffer, repeating centrifugation and wash twice, then taking the supernatant as a sample, and mixing well with an appropriate amount of 10× Annexin V Binding Buffer;

(2) Placing in a flow cytometer for measuring the cell activity state of each sample solution.

In one embodiment of the present disclosure, the buffer includes phosphate buffer.

In one embodiment of the present disclosure, the buffer comprises (8-28)% v/v sodium dihydrogen phosphate and (72-92)% v/v disodium hydrogen phosphate.

In one embodiment of the present disclosure, the pH of the buffer is 7.3-7.6.

In one embodiment of the present disclosure, the dilution volume ratio of the buffer to the aerobic denitrifier is 4-10:1.

In one embodiment of the present disclosure, a nylon membrane having a pore size of 6-15 μm is used for filtration.

In one embodiment of the present disclosure, the centrifugation speed is 5000-10000 rpm.

In one embodiment of the present disclosure, the mixed volume ratio of the sample supernatant to the 10× Annexin V Binding Buffer is 1:2-4.

In one embodiment of the present disclosure, the measurement of the cell activity state of each sample solution by the flow cytometer is carried out by adding 0.5 μl PI staining agent to the control FITC Annexin V group, adding 0.5 μl FITC Annexin V to the control PI group, adding 0.5 μl FITC Annexin V and 0.5 μl PI to the test group, mixing well, incubating in the dark at room temperature, and then testing on a flow cytometer.

In one embodiment of the present disclosure, the incubation time is 10-20 min.

A second object of the present disclosure is to provide a method for rapidly initiating the aerobic denitrifier engineering, which comprises: using the above method to determine the optimum preservation temperature; and placing the mature aerobic denitrifiers in a preservation medium for storage at an optimum preservation temperature, and using for wastewater treatment after recovering the activity.

In one embodiment of the present disclosure, the preservation medium of the aerobic denitrifier has a COD of 100 to 150 mg/L, NH₄⁺—N of 27.5-38.5 mg/L, and NO₃⁻—N of 4.5-8.0 mg/L.

In an embodiment of the present disclosure, the activity recovery of the aerobic denitrifier comprises inoculating the polyurethane filler enriched with the aerobic denitrifiers into a bioreactor. The effective volume of the bioreactor is 8.0˜20.0 L, and the DO(Dissolved Oxygen) of the bioreactor is controlled to 4-5 mg/L, the HRT (Hydraulic Retention Time) is 4-8 h, and the filling ratio of the polyurethane suspension filler is 30-40%.

A third object of the present disclosure is to apply the above method to wastewater treatment.

### Advantageous Effects of the Present Disclosure

The present disclosure characterizes the proportion of living cells, early apoptotic cells, late apoptotic cells and dead cells of various biofilms through flow cytometry, performs correlation analysis on the characteristic indexes of the aerobic denitrifier activity recovery process, and establishes the method for determining the optimum preservation temperature of the aerobic denitrifier based on the flow cytometry. By using the method, the step of recovering the aerobic denitrifier activity can be omitted, the wastewater treatment plant, which intends to adopt the aerobic denitrifier process technology to achieve efficient removal of nitrate nitrogen and total nitrogen, is effectively helped to realize the energy saving, consumption reducing operation, and the removal rate of nitrate nitrogen and total nitrogen can reach 90% and 88% respectively. At the same time, the starting time of engineering application of the aerobic denitrifier process can be effectively shortened, the long-term stable operation of the aerobic denitrifier process is maintained, and the method has high industrial feasibility.

### BRIEF DESCRIPTION OF FIGURES

FIG. 1 shows the removal rate of nitrate nitrogen in aerobic denitrifiers after activity recovery.

FIG. 2 shows the removal rate of total nitrogen in aerobic denitrifiers after activity recovery.

### DETAILED DESCRIPTION

### Example 1: Determination of the Optimum Preservation Temperature of Aerobic Denitrifiers

### Preservation Conditions of Aerobic Denitrifiers

The preservation temperature of the aerobic denitrifier was set to −20° C., 4° C. and 20° C. About 180 of the polyurethane filler enriched with the aerobic denitrifiers were taken out, divided into three equal portions and placed in 1000 mL serum bottles containing 600 mL of preservation medium, respectively. The preservation medium was the effluent from the anoxic tank of the sewage treatment plant, and had a COD of 100-150 mg/L, NH₄⁺—N of 27.5-38.5 mg/L, NO₃⁻—N of 4.5-8.0 mg/L. Serum bottles (3 parallel samples at each preservation temperature) were placed at −20° C., 4° C. and 20° C., and stored statically in the dark.

### Cell State Characterization of Aerobic Denitrifiers

Aerobic denitrifiers stored at −20° C., 4° C. and 20° C. were stored for more than 60 days, and then used to determine the aerobic denitrifier cell status. Cell state test conditions by flow cytometry were as follows:

(1) Crushed sample preparation 180 of the polyurethane filler enriched with the aerobic denitrifier (volume was about 200-300 ml) was taken, diluted to 1 L with phosphate buffer of pH 7.5, and used an ultrasonic breaker (ultrasonic energy density 90 kJ/g) to release the aerobic denitrifier in the polyurethane suspension filler into the phosphate buffer and ensure its uniform distribution;

(2) The crushed sample was filtered through a nylon membrane having a pore size of 10 μm, and then centrifuged at 8000 rpm for 5 min;

(3) Placed the sediment in a 50 ml centrifuge tube, and centrifuged at 8000 rpm for 5 min;

(4) The supernatant of the sample after centrifugation was pipetted with leaving about 0.1 mL of sample, the cells were purged with pre-cooled phosphate buffer (pH was 7.8), and the centrifugation and wash were repeated twice;

(5) The supernatant of the sample after centrifugation was pipetted with leaving about 0.1 mL of sample, and mixed well with 0.3 mL of 10× Annexin V Binding Buffer;

(6) 0.5 μL of PI staining agent was added to the control FITC Annexin V group, 0.5 μL of FITC Annexin V was added to the control PI group, 0.5 μL of FITC Annexin V and 0.5 μL of PI were added to the test group, which were mixed well and incubated for 15 min at room temperature in the dark, and then tested on a flow cytometer.

The cell state results were shown in Table 1. The living cell proportion of aerobic denitrifiers in the pilot system was higher, indicating that the aerobic denitrifier pilot system works well. The aerobic denitrifier stored at −20° C. had the lowest living cell content, indicating that it was not suitable to store aerobic denitrifiers at −20° C. The aerobic denitrifier stored at 4° C. had a proportion of living cell of about 57.7%, late apoptotic cells and dead cells of about 29.0%. The higher ratio of late apoptotic cells to dead cells indicated that 4° C. is not suitable for the preservation of aerobic denitrifiers. However, when the preservation temperature was 20° C., the living cell proportion of aerobic denitrifiers was as high as 66.0%, which was only 25.4% lower than that of the aerobic denitrifier in the pilot system, and the dead cells proportion was about 14.5%, indicating that 20° C. is the storage temperature with the lowest proportion of dead cells. Therefore, it was preliminarily determined that 20° C. was the optimum temperature for storing aerobic denitrifiers.

TABLE 1
Cell activity states (%) of aerobic denitrifiers after storage

| | | Early | Late | |
|---|---|---|---|---|
| aerobic | Living | apoptotic | apoptotic | |
| denitrifiers | cells | cells | cells | Dead cells |
| Pilot system | 88.5 ± 4.9 | 4.7 ± 0.1 | 3.3 ± 0.1 | 3.5 ± 0.1 |
| Stored at −20° C. | 45.5 ± 2.2 | 12.5 ± 0.8 | 9.9 ± 0.5 | 32.1 ± 1.7 |
| Stored at 4° C. | 57.7 ± 3.0 | 13.3 ± 0.8 | 12.5 ± 0.7 | 16.5 ± 0.5 |
| Stored at 20° C. | 66.0 ± 3.5 | 12.5 ± 0.7 | 7.0 ± 0.3 | 14.5 ± 0.4 |

### Example 2: Verification of the Tested Optimum Temperature Results of Aerobic Denitrifiers

### Activity Recovery Conditions of the Stored Aerobic Denitrifiers

The polyurethane filler enriched with the aerobic denitrifier derived from different serum bottles was inoculated into a bioreactor for the activity recovery of aerobic denitrifiers; the aerobic denitrifier stored at −20° C., 4° C. and 20° C. was placed in R1 (bioreactor 1), R2 (bioreactor 2) and R3 (bioreactor 3), respectively. The bioreactor had an effective volume of 10.0 L, and the DO of the bioreactor was controlled to 4-5 mg/L, the HRT was controlled to 4-8 h, and the filling ratio of the polyurethane suspension filler was 35%.

### Characteristics of Aerobic Denitrifiers After Activity Recovery

After 30 days of activity recovery, the characteristics of aerobic denitrifiers in R1, R2 and R3 were shown in Table 2. As shown in Table 2, after recovering the aerobic denitrifier activity, the nitrate removal rate and total nitrogen removal rate in R1 and R2 are lower than the nitrate removal rate and total nitrogen removal rate of the aerobic denitrifier before storage, only the nitrate removal rate and total nitrogen removal rate of the aerobic denitrifier stored at 20° C. were more consistent with those before storage. The denitrification rate and simultaneous nitrification and denitrification rate of aerobic denitrifiers stored at different storage temperatures were lower than before storage. After activity recovery, the denitrification rate and simultaneous nitrification and denitrification rate of aerobic denitrifiers in R2 and R3 were closer to those before storage, but the denitrification rate and simultaneous nitrification and denitrification rate of aerobic denitrifiers in R1 were relatively low.

Generally, the denitrification rate and simultaneous nitrification and denitrification rate of aerobic denitrifiers were 6.0 mg/(m2·h) and 1.5 mg/(m2·h), respectively, and the domesticated aerobic denitrifiers in the pilot plant will respectively take 21 d and 30 d to reach the same denitrification rate and simultaneous nitrification and denitrification rates.

After the activity of the stored aerobic denitrifier was recovered, the aerobic denitrifier in R1 will respectively take 18 d and 25 d to reach the same denitrification rate and simultaneous nitrification and denitrification rates, the aerobic denitrifier in R2 will respectively take 15 d and 20 d to reach the same denitrification rate and simultaneous nitrification and denitrification rates, and the aerobic denitrifier in R3 will respectively take 11 d and 18 d to reach the same denitrification rate and simultaneous nitrification and denitrification rates, indicating that the aerobic denitrifier after the activity recovery all had better nitrogen removal effects, wherein the aerobic denitrifier stored at the temperature of 20° C. has the shortest activity recovery time and the condition at 20° C. was more suitable for storing the aerobic denitrifier.

TABLE 2
Properties of aerobic denitrifiers after
preservation and activity recovery

| | | | Time (d) | Time (d) required |
|---|---|---|---|---|
| | | | required for | for simultaneous |
| | nitrate | total | denitrifi- | nitrification and |
| | nitrogen | nitrogen | cation | denitrification |
| | removal | removal | rate to be | rate to be more |
| | rate | rate | more than 6.0 | than 1.5 |
| | (mg/h) | (mg/h) | mg/(m² · h) | mg/(m² · h) |
| Before | 4.5 | 5.8 | 21 | 30 |
| preservation | | | | |

After aerobic denitrifiers preservation

| | | | | |
|---|---|---|---|---|
| After | 2.0 | 2.7 | — | — |
| preservation | | | | |
| at −20° C. | | | | |
| After | 2.1 | 2.9 | — | — |
| preservation | | | | |
| at 4° C. | | | | |
| After | 2.5 | 3.1 | — | — |
| preservation | | | | |
| at 20° C. | | | | |

After activity recovery of the aerobic denitrifier

| | | | | |
|---|---|---|---|---|
| Aerobic | 3.5 | 4.5 | 18 | 25 |
| denitrifiers | | | | |
| stored | | | | |
| at −20° C. | | | | |
| Aerobic | 4.1 | 5.1 | 15 | 20 |
| denitrifiers | | | | |
| stored | | | | |
| at 4° C. | | | | |
| Aerobic | 4.3 | 5.7 | 11 | 18 |
| denitrifiers | | | | |
| stored | | | | |
| at 20° C. | | | | |

### Removal Efficiency of Pollutants by the Aerobic Denitrifier After Activity Recovery

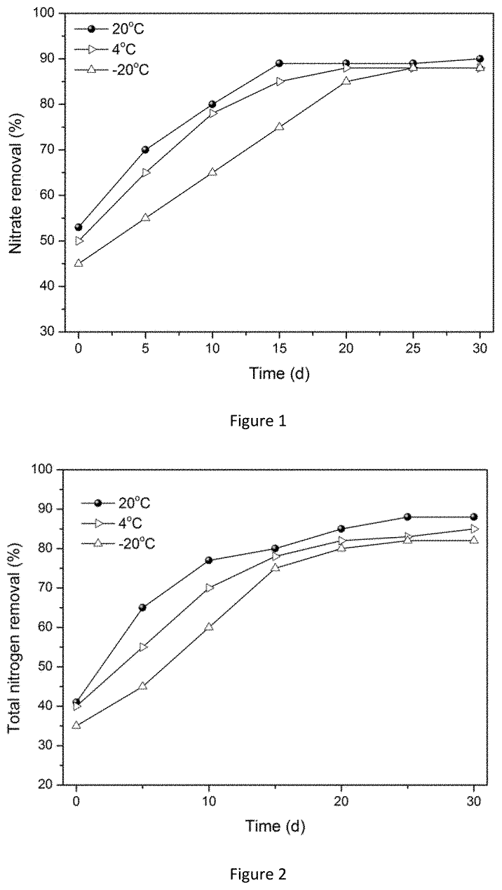

After the activity recovery process, the removal rates of nitrate and total nitrogen by aerobic denitrifiers at different preservation temperatures were gradually increased (FIG. 1 and FIG. 2), and the removal rates of nitrate and total nitrogen were over 90% and 88%, respectively. On the 18^{th} day after the activity recovery, the aerobic denitrifier in R3 had the best removal effect on nitrate and total nitrogen, and the nitrate and total nitrogen removal rates showed a steady increase trend. This result also corresponded to the fastest recovery of the higher denitrification rate and simultaneous nitrification and denitrification rate by the aerobic denitrifier in R3 in Table 2, indicating that the condition at 20° C. was more suitable for storing aerobic denitrifiers and was highly feasible in practical applications.

### Correlation Between Aerobic Denitrifiers Characteristics and Cell States After Activity Recovery

After 30 d of aerobic denitrifiers activity recovery, flow cytometry was used to analyze the aerobic denitrifier cell states (as shown in Table 3). The living cell content in aerobic denitrifiers at different preservation temperatures was basically the same as the content of living cells in the aerobic denitrifier of the pilot system, indicating that all of the aerobic denitrifier after the activity recovery can play the role of nitrate and total nitrogen removal. Among them, the proportion of aerobic denitrifiers living cells in R3 was the highest (80.3%±4.2%), and the proportion of late apoptotic cells (7.5%±0.3%) and the proportion of dead cells (6.7%±0.1%) were the lowest, indicating the aerobic denitrifier cells stored at 20° C. had the highest cell activity and 20° C. was more suitable as a condition for storing aerobic denitrifiers.

TABLE 3
Cell activity states (%) of aerobic denitrifier
cells after activity recovery (30 d)

| | | Early | Late | |
|---|---|---|---|---|
| Aerobic | Living | apoptotic | apoptotic | |
| denitrifier | cells | cells | cells | Dead cells |
| Pilot system | 87.0 ± 4.5 | 4.5 ± 0.1 | 3.6 ± 0.1 | 4.9 ± 0.2 |
| Stored at −20° C. | 78.0 ± 4.3 | 6.0 ± 0.1 | 7.1 ± 0.1 | 8.9 ± 0.3 |
| Stored at 4° C. | 78.5 ± 4.3 | 6.0 ± 0.1 | 7.5 ± 0.3 | 9.0 ± 0.3 |
| Stored at 20° C. | 80.3 ± 4.2 | 5.5 ± 0.1 | 7.5 ± 0.3 | 6.7 ± 0.1 |

According to Correl correlation analysis, it was found that the denitrification rate and simultaneous nitrification and denitrification rate of aerobic denitrifier had a very high correlation with the proportion of aerobic denitrifier live cells (as shown in Table 4), and the correlation coefficients were 0.9088 and 0.9507, respectively, indicating that the use of the proportion of aerobic denitrifier living cells as a method for evaluating the activity of aerobic denitrifier was extremely feasible. At the same time, in the stored aerobic denitrifier, the proportion of aerobic denitrifier live cells was the highest under the preservation condition of 20° C., which was consistent with results for the proportion of aerobic denitrifier living cells in R3 after activity recovery.

TABLE 4
Correlation between aerobic denitrifier characteristics
and cell activity sates after activity recovery (30 d)

| | Aerobic | Aerobic | Aerobic |
|---|---|---|---|
| | denitrifier | denitrifier | denitrifier |
| | stored | stored | stored |
| | at −20° C. | at 4° C. | at 20° C. |
| Denitrification | 5.5 | 5.8 | 6.0 |
| rate | | | |
| mg/(m² · h) | | | |
| Simultaneous | 1.0 | 1.2 | 1.2 |
| nitrification and | | | |
| denitrification | | | |
| rate | | | |
| mg/(m² · h) | | | |
| Living cell | 78.0 ± 4.3 | 78.5 ± 4.3 | 80.3 ± 4.2 |
| proportion (%) | | | |

| | |
|---|---|
| Correlation | 0.9088 |
| between | |
| denitrification | |
| rate and living | |
| cells | |
| Correlation between | 0.9507 |
| simultaneous | |
| nitrification and | |
| denitrification | |
| rate with living | |
| cells | |

Therefore, it was determined that 20° C. was the most suitable condition for storing aerobic denitrifier, and flow cytometry can be used as the basis for determining the optimum preservation temperature of aerobic granular sludge. Flow cytometry is easy to operate, the data are fast and easy to obtain, accurate and reliable, and the aerobic denitrifier activity recovery process can be omitted, which is of great significance for the preservation and activity recovery of aerobic denitrifier.

### Comparative Example 1

### Preservation and Culture of Nitrifying Denitrifying Biofilm

### Preservation Conditions of Aerobic Denitrifiers

The preservation temperature of the aerobic denitrifier was set to −20° C., 4° C. and 20° C. About 180 of the polyurethane filler enriched with the aerobic denitrifier were taken out, divided into three equal portions and placed in 1000 mL serum bottles containing 600 mL of preservation medium, respectively. The preservation medium was the effluent from the anoxic tank of the sewage treatment plant, and had a COD of 100-150 mg/L, NH₄⁺—N of 27.5-38.5 mg/L, NO₃⁻—N of 4.5-8.0 mg/L. Serum bottles (3 parallel samples at each preservation temperature) were placed at −20° C., 4° C. and 20° C., and stored statically in the dark.

### Cell State Characterization of Aerobic Denitrifiers

Aerobic denitrifiers stored at −20° C., 4° C. and 20° C. were stored for more than 60 days, and then used to determine the aerobic denitrifier cell status. Cell state test conditions by flow cytometry were as follows:

(1) Crushed sample preparation 180 of the polyurethane filler enriched with the aerobic denitrifier (volume was about 200-300 ml) was taken, diluted to 1 L with phosphate buffer of pH 7.2 and 7.8, respectively, and used an ultrasonic breaker (ultrasonic energy density 90 kJ/g) to release the aerobic denitrifier in the polyurethane suspension filler into the phosphate buffer and ensure its uniform distribution;

(2) The crushed sample was filtered through a nylon membrane having a pore size of 10 μm, and then centrifuged at 8000 rpm for 5 min;

(3) Placed the sediment in a 50 ml centrifuge tube, and centrifuged at 8000 rpm for 5 min;

(4) The supernatant of the sample after centrifugation was pipetted with leaving about 0.1 mL of sample, the cells were purged with pre-cooled phosphate buffer (pH was 7.8), and the centrifugation and wash were repeated twice;

(5) The supernatant of the sample after centrifugation was pipetted with leaving about 0.1 mL of sample, and mixed well with 0.3 mL of 10× Annexin V Binding Buffer;

(6) 0.5 μL of PI staining agent was added to the control FITC Annexin V group, 0.5 μL of FITC Annexin V was added to the control PI group, 0.5 μL of FITC Annexin V and 0.5 μL of PI were added to the test group, which were mixed well and incubated for 15 min at room temperature in the dark, and then tested on a flow cytometer.

The cell state results were shown in Table 5 and Table 6.

TABLE 5
Cell activity states (%) of aerobic denitrifiers
after storage (phosphate buffer of pH 7.2)

| | | Early | Late | |
|---|---|---|---|---|
| aerobic | Living | apoptotic | apoptotic | |
| denitrifiers | cells | cells | cells | Dead cells |
| Pilot system | 88.5 ± 4.9 | 4.7 ± 0.1 | 3.3 ± 0.1 | 3.5 ± 0.1 |
| Stored at −20° C. | 52.5 ± 3.1 | 6.3 ± 0.5 | 11.7 ± 0.8 | 29.5 ± 1.4 |
| Stored at 4° C. | 57.0 ± 3.0 | 3.2 ± 0.2 | 13.8 ± 0.8 | 26.0 ± 1.3 |
| Stored at 20° C. | 59.7 ± 3.1 | 3.1 ± 0.2 | 17.9 ± 1.0 | 19.3 ± 1.0 |

As shown in Table 5, aerobic denitrifiers stored at 20° C. had the highest proportion of live cells, but aerobic denitrifiers at three temperatures had relatively close viable cell content, and the total content of late apoptotic and dead cells was also relatively close, so when the value of pH is 7.2, the significance of flow cytometric analysis is poor, and it is not suitable as a suitable pH for determining the optimal storage temperature of aerobic denitrifiers.

TABLE 6
Cell activity states (%) of aerobic denitrifiers
after storage (phosphate buffer of pH 7.8)

| | | Early | Late | |
|---|---|---|---|---|
| aerobic | Living | apoptotic | apoptotic | |
| denitrifiers | cells | cells | cells | Dead cells |
| Pilot system | 88.5 ± 4.9 | 4.7 ± 0.1 | 3.3 ± 0.1 | 3.5 ± 0.1 |
| Stored at −20° C. | 49.2 ± 2.7 | 0.5 ± 0.1 | 21.5 ± 1.7 | 27.6 ± 1.9 |
| Stored at 4° C. | 47.9 ± 2.5 | 0.2 ± 0.1 | 22.9 ± 1.8 | 29.0 ± 2.3 |
| Stored at 20° C. | 48.9 ± 2.5 | 0.3 ± 0.1 | 21.8 ± 1.9 | 29.0 ± 2.1 |

As shown in Table 6, the aerobic denitrifiers stored at three temperatures not only had a relatively close content of live cells, but also had a similar content of late apoptotic cells and dead cells. At the same time, the results based on the detection of early apoptosis cells were low, indicating that when the value of pH is 7.2, the results of flow cytometric analysis could not effectively determine the optimal storage temperature for aerobic denitrifiers.

What is claimed is:

1. A method for determining an optimum preservation temperature of an aerobic denitrifier, comprising: measuring a cell activity state of the aerobic denitrifier stored at different temperatures based on flow cytometry, and taking a preservation temperature closest to the cell activity state of the aerobic denitrifier during a pilot operation as the optimum preservation temperature, wherein the measuring the cell activity state comprises measuring contents of living cells, early apoptotic cells, late apoptotic cells and dead cells.

2. The method according to claim 1, wherein the measuring the cell activity state of the wastewater treatment biofilm based on the flow cytometry comprises:
(1) preparing a test sample solution of the aerobic denitrifier: diluting an aerobic denitrifier sample with a buffer, mixing evenly, filtering, centrifuging, leaving a supernatant, purging the cells with a pre-cooled phosphate buffer, repeating centrifugation and wash twice, then taking the supernatant as a sample, and mixing well with an appropriate amount of 10× Annexin V Binding Buffer; and
(2) placing in a flow cytometer for measuring a cell activity state of each sample solution.

3. The method according to claim 2, wherein a pH value of the buffer is 7.3 -7.6.

4. The method according to claim 2, wherein the buffer includes a phosphate buffer.

5. The method according to claim 3, wherein the buffer comprises (8-28)% v/v sodium dihydrogen phosphate and (72-92)% v/v disodium hydrogen phosphate.

6. The method according to claim 3, wherein a dilution volume ratio of the buffer to the aerobic denitrifier is 4-10:1.

7. The method according to claim 3, wherein a nylon membrane having a pore size of 6-15 μm is used for filtration.

8. The method according to claim 4, wherein a nylon membrane having a pore size of 6-15 μm is used for filtration.

9. A method for rapidly initiating aerobic denitrifier engineering, comprising: using the method according to claim 1 to determine an optimum preservation temperature; and placing a mature aerobic denitrifier in a preservation medium for storage at the optimum preservation temperature, and using for the aerobic denitrifier engineering after recovering activity.

10. The method according to claim 9, wherein the preservation medium has 100 to 150 mg/L of COD, 27.5-38.5 mg/L of NH₄⁺—N, and 4.5-8.0 mg/L of NO₃⁻—N.
